(19) United States
(12) Patent Application Publication
(10) Pub. No.: US 2008/0238445 A1
(43) Pub. Date: Oct. 2, 2008

(54) Measuring Device For Measuring the State of Oils or Fats

(76) Inventors: Mike Muhl, Freiburg (DE); Juergen Hall, Friedenweiler (DE)

Correspondence Address: MUIRHEAD AND SATURNELLI, LLC, 200 FRIBERG PARKWAY, SUITE 1001, WESTBOROUGH, MA, 01581, US

(21) Appl. No.: 11/547,894

(22) PCT Filed: Mar. 30, 2005
(86) PCT No.: PCT/EP2005/003323

§ 371 Date: Apr. 21, 2008

(30) Foreign Application Priority Data
Apr. 6, 2004 (DE) 10 2004 016 955.1

(51) Int. Cl. G01N 33/28; G01R 27/26

(52) U.S. Cl. 324/663

(57) ABSTRACT

A measuring device is used to measure the state of oils or fats. Said measuring device comprises a housing, a hollow connecting element which is secured therein and a carrier which is applied to the opposite end of the connecting element, said carrier being used to receive a sensor which can be used to measure the electric property of the product which is to be measured, in addition to a sensor which can be used to measure the temperature of the product which is to be measured. The two sensors are in contact with the evaluation system by means of at least one electric line, which is arranged in the region of the housing and/or on the end of the connecting element oriented towards the housing. The temperature sensor is arranged at a pronounced distance from the connecting element and the sensor in order to measure the electric property of the product which is to be measured.

The present invention relates to a measuring device for measuring the state of oils or fats according to the definition of the species in Claim 1.

Hot oils or fats are often used not only once, but are utilized in deep fryers over a longer period for successively preparing different foods. The oil or fat is decomposed by oxidation at the hot operating temperatures between approximately 120° C. and 180° C. and undesirable chemical products such as free fatty acids and polymers are formed, which not only impair the taste, but may also have adverse health effects.

In order to avoid replacing frying oils or fats too early or too late, measuring devices are used for measuring the state of oils or fats, including tests for their electrical properties. Measuring the dielectric constant, which is a reliable measure of the degree of decomposition of the fats or oils, is particularly suitable.

EP 1 046 908 A2, for example, describes a measuring device for measuring the state of an oil or fat, which has a housing which contains the electronic analyzer units and a data display, as well as a tubular connecting element with a sensor situated at its tip which may be directly immersed into the hot oil or fat and is suitable for determining the dielectric constant. The sensor and the electronic analyzer circuit are electrically connected via a cable laid freely within the connecting element.

DE 101 63 760 A1 describes a refinement of the above-mentioned measuring device. In the measuring device presented therein, the electrical conductors between the sensor and the electronic analyzer circuit are formed by metallic conductors printed on a ceramic substrate. The tubular connecting element is shaped in such a way that it surrounds most of the substrate and narrows downward in such a way that only the area of the substrate on which the sensor is situated is accessible from the outside. An insulating sealing adhesive is introduced and cured between the substrate and the connecting element, so that there is no electrical connection between the connecting element and the electrical conductors. In addition, good insulation is thus achieved, which prevents the oil from penetrating inside the connecting element. A temperature sensor whose measurement results may also be processed by the electronic analyzer circuit may also be provided on the ceramic substrate in the immediate proximity of the lower end of the connecting element.

The disadvantage of this device, however, is the inertia of the temperature sensor in its response to temperature changes. Therefore, when the sensors of the measuring device are immersed into the oil or fat, it is necessary either to wait a few minutes until the temperature sensor displays the actual temperature of the oil, or to determine the actual value of the oil temperature by extrapolating from the temperature increase curve. This results in either undesirably long waiting times or inaccuracy of the measured value caused by the extrapolation.

The object of the present invention is therefore to provide a measuring device for measuring the state of oils or fats which has minimum response time after immersion into the medium to be measured and creates the possibility of continuous measurements.

This object is achieved by the features of Claim 1. Embodiments and refinements of the inventive idea are the subject matter of the subclaims.

Due to the fact that the temperature sensor is situated at a certain distance from the connecting element, which has poor thermal conductivity, the temperature sensor has a very small “thermal mass,” so that it may quickly respond to temperature changes, making accurate temperature measurements possible at all times.

The temperature sensor is preferably situated in the front end area of the substrate, which prevents interference from other components.

The temperature sensor preferably has the smallest possible dimensions for short response time.

The sensor designed as an interdigital capacitor for measuring the dielectric constant preferably has the same response speed as the temperature sensor, so that the response speed of the overall system increases.

It is particularly preferable if the two sensors are situated on opposite sides at the front end of the substrate, which ensures that both sensors are always exposed to the same ambient temperature and thus no measuring inaccuracies occur.

Further details, features, and advantages of the present invention are derived from the description that follows and the appended drawing.

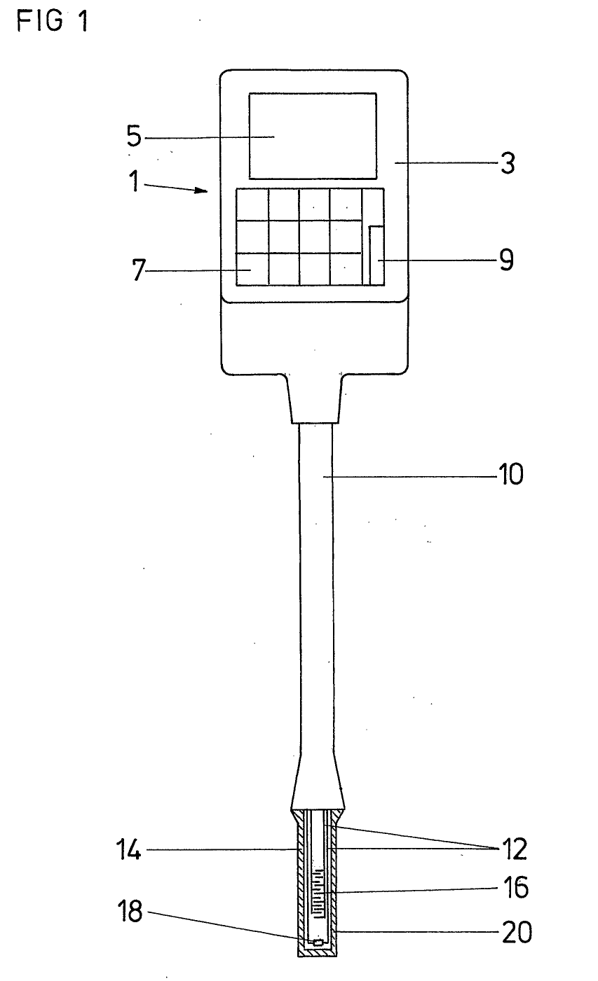

FIG. 1 shows a specific embodiment of the measuring device according to the present invention in a front view;

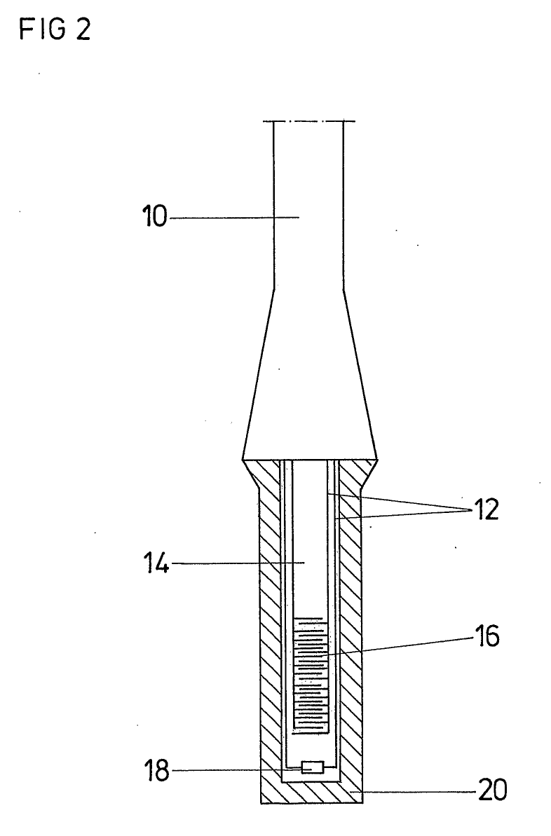

FIG. 2 shows an enlarged view of the lower area, to be immersed, of the measuring device of FIG. 1, and

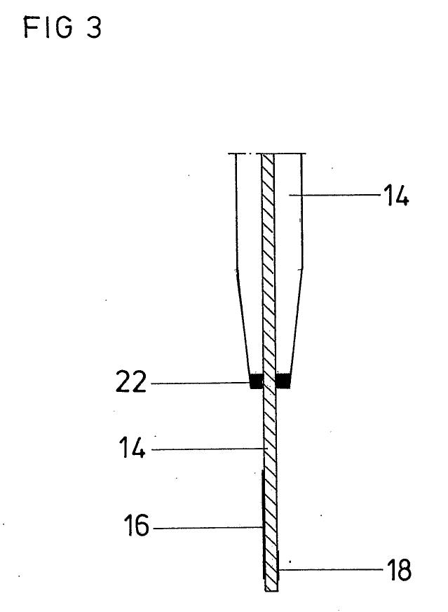

FIG. 3 shows a longitudinal section through the lower area of a second specific embodiment of the measuring device according to the present invention.

FIG. 1 shows a measuring device 1 according to the present invention for measuring the state of oils or fats, which has a housing 3 in its upper area. The housing has a display 5 for displaying measured values. The display is preferably designed as an LCD display and is switchable between graphic display, e.g., color coding of the measured values, and numerical display. A keyboard 7 is provided for inputting control instructions, via which instructions may be issued to the central control unit (not shown). Keyboard 7 is preferably designed as a membrane keyboard. The housing may preferably also have an interface 9, which may be used for communication with external computers. Measuring device 1 is preferably designed to be self-adjusting. During the use of measuring device 1, housing 3 is simultaneously used as a handle for the operator.

A hollow connecting element 10, which is sufficiently long and is made of a material with poor thermal conductivity, protrudes downward from housing 3, so that the sensitive electronic analyzer circuit (not shown) of measuring device 1, which is located in the area of housing 3 and/or in the area of connecting element 10 facing housing 3, is adequately protected against the heat of the oil or fat to be measured. These measures ensure that the operator is able to safely perform the measurements. Connecting element 10 is preferably made of stainless steel, which, in addition to its poor thermal conductivity, is also suitable because of its unrestricted applicability in the food industry. Connecting element 10 is preferably designed as a tubular component and is suitable for receiving electrical conductors 12 running inside connecting element 10. Electrical conductors 12 are situated on at least one flat substrate 14, which is characterized by its insulating properties, for example, on a substrate 14 made of a ceramic material.

In the lower area of substrate 14, there is a sensor 16 for measuring electrical properties of the oil or fat and a temperature sensor 18, whose measured values are conducted via electrical conductors 12 on substrate 14 to the electronic analyzer circuit. A protective means 20 for protecting sensors 16, 18 against external influences, in particular against contact with the bottom or the walls of the measuring container, may be applied around the lower area of substrate 14. In the present case, protective means 20 is designed as a peripheral edge of flat substrate 14, connected to connecting element 10.

The gap between substrate 14 and connecting element 10 is insulatingly sealed via suitable sealants 22 as is apparent from FIG. 3. In the lower end area of connecting element 10, a suitable adhesive 22, for example, a silicone adhesive, is injected into the gap between substrate 14 and connecting element 10, so that these are not in direct contact and are thus insulated from one another. At the same time, adhesive 22 functions as a seal of connecting element 10, so that no oil or fat is able to penetrate into the inside of connecting element 10. The adhesive surface must reliably prevent water inclusions; otherwise an explosion risk, as well as contamination of the oil or fat to be measured, may result. Substrate 14 as a single-piece element may extend to the electronic analyzer circuit; it may, however, also be isolated by placing a plurality of substrate sections together in a row via suitable conductive connecting means. This arrangement provides special advantages regarding the heat load on the electronic analyzer circuit.

FIG. 2 shows an enlarged view of the lower sections of connecting element 10 and of substrate 14, which are suitable for being immersed into the liquid to be measured. Sensor 16 for measuring the dielectric constant has a capacitor which measures the dielectric constant of the oil. It is preferably designed as an interdigital capacitor which has fine intermeshing gold wires, each of which continues as an electrical conductor 12 leading to the electronic analyzer circuit. Conductors 12 may be made of a fine plating of gold or copper on substrate 14, the plating being printed directly onto the ceramic component. A multilayer construction of substrate 14 is also conceivable, which may better protect conductors 12 against environmental influences.

Temperature sensor 18 is designed, for example, as an electrical resistor, which may be made of platinum, for example, or another suitable material. Temperature sensor 18 has a very small thermal mass due to its greatest possible distance from connecting element 10. In addition, it is applied directly to ceramic substrate 14 in thick-layer or thin-layer technology and with the smallest possible dimensions. If both sensors 16, 18 are set to the same response speed, the response speed of the overall system is considerably increased, which makes continuous measurement possible and extrapolation to an end value of the dielectric constant measurement unnecessary.

Thus, in the case of “optimal frying,” usable fresh oil may be admixed in a controlled manner in such a way that the proportion of polar components may be set between 12% and 18%, for example, and the result may be monitored directly during mixing due to the quick adjustment of measuring device 1.

As FIG. 3 shows, the temperature sensor may also be situated on the opposite side of substrate 14 in the area of the tip of substrate 14, which makes it possible to further reduce the size of the measuring device, while exposing both sensors 16, 18 to the same ambient temperature.

In addition to the specific embodiments shown, many other exemplary embodiments of the present invention are conceivable in which similar geometric arrangements of the individual components are used.

1. A measuring device for measuring the state of oils or fats, comprising:
a housing;
a hollow connecting element attached to the housing; and
a substrate mounted on the opposite end of the connecting element for receiving a sensor for measuring an electrical property of oil or fat to be measured and a temperature sensor for measuring a temperature of the oil or fat to be measured, the two sensors being connected to an electronic analyzer circuit via at least one electrical conductor, the electronic analyzer circuit being situated in an area of at least one of the housing an end of the connecting element facing the housing, wherein the temperature sensor is situated at least at the same distance from the connecting element as the sensor for measuring an electrical property of the oil or fat to be measured.

2. The measuring device as recited in claim 1, wherein the temperature sensor is situated in a front end area of the substrate.

3. The measuring device as recited in claim 1, wherein the temperature sensor has smaller dimensions than the sensor for measuring an electrical property of the oil or fat to be measured.

4. The measuring device as recited in claim 1, wherein the sensor measures a dielectric constant of the oil or fat to be measured.

5. The measuring device as recited in claim 1, wherein the sensor is designed as an interdigital capacitor.

6. The measuring device as recited in claim 1, wherein both sensors have the same response speed.

7. The measuring device as recited in claim 1, wherein both sensors are situated on opposite sides in a front end area of the substrate.

8. The measuring device as recited in claim 1,
wherein the temperature sensor has smaller dimensions than the sensor for measuring an electrical property of the oil or fat to be measured,
wherein the sensor for measuring an electrical property of the oil or fat to be measured measures a dielectric constant of the oil or fat to be measured, and
wherein both sensors have the same response speed.

9. A measuring device for measuring the state of oils or fats, comprising:
a housing;
a connecting element attached to the housing;
an analyzer circuit disposed in an area of at least one of the housing and an end of the connecting element facing the housing;
a sensor for measuring an electrical property of oil or fat to be measured and that is coupled to the analyzer circuit;
a temperature sensor for measuring a temperature of the oil or fat to be measured and that is coupled to the analyzer circuit, wherein the temperature sensor is disposed at least at the same distance from the connecting element as the sensor for measuring an electrical property of the oil or fat to be measured.

10. The measuring device as recited in claim 9, wherein the temperature sensor has smaller dimensions than the sensor for measuring an electrical property of the oil or fat to be measured.

11. The measuring device as recited in claim 9, wherein the sensor for measuring an electrical property of the oil or fat to be measured measures a dielectric constant of the oil or fat to be measured.

12. The measuring device as recited in claim 9, wherein the sensor for measuring an electrical property of the oil or fat to be measured is an interdigital capacitor.

13. The measuring device as recited in claim 9, wherein both sensors have the same response speed.
